(19) United States
(12) Patent Application Publication
(10) Pub. No.: US 2005/0163761 A1
(43) Pub. Date: Jul. 28, 2005

(54) Agent for inducing or inhibiting an angiogenesis

(76) Inventor: Hans-Gunther Machens, Lubeck (DE)

Correspondence Address: Ronald R Santucci;Frommer Lawrence &Haug, 745 Fifth Avenue, New York, NY, 10151, US

(21) Appl. No.: 10/505,236

(22) PCT Filed: Feb. 20, 2003
(86) PCT No.: PCT/EP03/01766

(30) Foreign Application Priority Data
Feb. 21, 2002 (DE) 102 07 436.4
May 7, 2002 (DE) 102 20 474.8

(51) Int. Cl. A61K 48/00

(52) U.S. Cl. 424/93.21

(57) ABSTRACT

The invention relates to an agent for inducing or inhibiting angiogenesis, to a method for the production thereof and to the use thereof.

The invention relates to an agent for inducing or inhibiting angiogenesis (formation of new blood vessels), to a method for the production thereof and to the use thereof.

A basic principle of all life is the maintenance of an intracellular and extracellular steady state and of a physiological dynamic equilibrium. Any interruption or disturbance in the preservation of that equilibrium ultimately endangers and destroys the life of the cell. In all relatively highly developed organisms, blood flow plays a central role in ensuring cell function. The provision of oxygen and high-energy substrates and the removal of metabolites have to be adapted to the constantly changing conditions. Physiological changes in tissue perfusion, whether involving an increase or a reduction, must be equivalent to the altered energy requirements of the cell and must on no account be interrupted. If that occurs, however, such a change will result sooner or later in destruction of the cell.

One way in which the organism can meet the cells' increased energy requirement, apart from opening vessels not hitherto included in the circulation, is to develop new microcirculatory pathways. The latter process is set in motion not when an abrupt interruption of the steady state occurs but, rather, when the increased energy requirement sets in sufficiently slowly, so that the organism still has time to develop new capillaries. That process is called angiogenesis and takes place, like any other cell division in the organism, from the first moment of embryonic life until the death of the organism as a whole. It involves a biological mechanism in which new capillaries are formed by activation, migration and proliferation of endothelial cells from pre-existing endothelial-pericyte associations. As the first step, outgrowths of the activated endothelial cells penetrate into the connective tissue stroma by partial disintegration of the basal membrane in the parent vessel. The migration of the endothelial cell is normally directionally determined by an angiogenetic stimulus and is assisted by a proliferation of the neighbouring endothelial cells. Following development of a lumen and fusion of two neighbouring outgrowing endothelial cells, blood flow through the newly formed capillaries commences. If the described angiogenetic processes are initiated by the body in good time, impending ischaemia, that is to say, insufficient blood supply to the dependent tissue parts of the body, can be avoided. In many cases, however, that mechanism is not sufficient, with the result that the ischaemically vulnerable tissue dies (e.g. cardiac infarct/acute ischaemia or diabetic ulcer/chronic ischaemia).

In the age of gene technology, there is a completely new therapeutic approach to that problem, namely the induction of neovascularisation by means of cell transduction for the production of angiogenetically active factors in the tissue affected.

Surprisingly, in plastic surgery, there have hitherto been only few experimental attempts at utilising the possibilities of gene transfer at all for that field. In the works that have so far appeared on the experimental and clinical transduction of cells for the production of angiogenetically active factors, no direct relevance to the field of plastic surgery has yet been identified. The possible therapeutic significance of such angiogenetically active substances in precisely that field of surgery would, however, surely be undisputed. Controlled influencing of wound healing and the survival of tissue as a result of vessel induction not only would have a direct clinical significance but also would establish a new scientific field for the study and better understanding of micro-circulatory phenomena in tissue under physiological and pathophysiological conditions.

On the other hand, it is also possible to find by gene technology anti-angiogenetic therapeutic approaches, namely when it is essential to reduce or even to interrupt circulation in the tissue. That, however, may be the case only when the tissue produced is not desired, as is the case with the formation of benign and malignant tumours. In that case, it is possible using antiangiogenetic measures to impair the circulation of such tumours in such a manner that the tumours ultimately become smaller or even die back completely. In oncology, there are a great many therapeutic approaches to that end, which, however, differ from the approach described herein.

According to the invention, an agent for inducing or inhibiting angiogenesis is proposed that comprises isogenic or autologous body cells that express at least one angiogenetic or anti-angiogenetic protein. Isogenic (=syngenic) cells in the sense of the invention are genetically identical cells which do not result in immuno-incompatibility after re-transplantation into an isogenic organism. In humans, isogenic cells are known in the form of homologous and genetically identical cells from monozygotic twins. Transplantation medicine relies in the case of an isogenic (=isogenetic) transplant on the rather rare genetic identity of donor and recipient, which, for example, also exists in the case of monozygotic twins. In the animal kingdom, especially in experimental animals, such as mice and rats, that come from true inbred strains, on the other hand, isogenic cells abound. Autologous cells are body cells where donor and recipient are one and the same organism.

According to one embodiment of the invention, in the agent according to the invention the at least one angiogenetic protein is selected from PDGF-A (platelet derived growth factor A), PDGF-B (platelet derived growth factor B), VEGF (vascular endothelial growth factor), bFGF (basic fibroblast growth factor), TGFbeta (tumour growth factor beta), angiopoetin 1 and angiopoetin.

According to another embodiment of the invention, in the agent according to the invention the at least one anti-angiogenetic protein is selected from VEGFR-1 (vascular endothelial growth factor receptor 1), angiostatin, endostatin and receptor blockers for VEGFR1 and VEGFR2.

The invention further provides a method for the production of an agent according to the invention, in which
a) the formation of isogenic or autologous cells is induced in a body by implantation of biologically inert material, e.g. Silastic,
b) the cells formed in step a) are extracted from the body,
c) the cells obtained in step b) are genetically modified, e.g. by retroviral, especially adenoviral, gene transfer, in such a manner that in the case of a desired induction of angiogenesis they express at least one angiogenetic protein or in the case of a desired inhibition of angiogenesis they express at least one anti-angiogenetic protein.

A biologically inert or metabolically inert material is a material that does not participate in metabolic processes of the body but that is nevertheless capable of causing a cellular reaction (accumulation of fibroblasts). When such a material (e.g. Silastic) is implanted, within a few days (5-7) a connective tissue capsule containing the desired fibroblasts forms around the implant. That capsule together with the fibroblasts can be removed and, after trypsinisation of the material, the fibroblasts can be isolated and cultured. Those fibroblasts may be isogenic. That is to say, they originate from an isogenic donor animal having the same genetic characteristics as the animals into which the genetically modified isogenic fibroblasts will subsequently be re-transplanted. The alternative (and the clinically most appropriate) is to extract the fibroblasts from the patient to whom those cells are subsequently to be re-transplanted again following genetic modification. In that case, autologous cells would be involved, that is to say, cells where the donor and the recipient are identical.

According to one embodiment of the method according to the invention, the cells obtained in step c) express an angiogenetic protein selected from PDGF-A (platelet derived growth factor A), PDGF-B (platelet derived growth factor B), VEGF (vascular endothelial growth factor), bFGF (basic fibroblast growth factor), TGFbeta (tumour growth factor beta), angiopoetin 1 and angiopoetin.

According to another embodiment of the method according to the invention, the cells obtained in step c) express an anti-angiogenetic protein selected from VEGFR-1 (vascular endothelial growth factor receptor 1), angiostatin, endostatin and receptor blockers for VEGFR1 and VEGFR2.

The invention also indicates the use of an agent according to the invention, or of an agent produced by a method according to the invention, for inducing or inhibiting angiogenesis.

According to one embodiment of the use according to the invention, an agent according to the invention or an agent produced by a method according to the invention is introduced into isogenic or autologous tissue of the body in which angiogenesis is to be induced or inhibited.

The invention further relates to the use of the agent comprising isogenic or autologous body cells that express at least one angiogenetic or anti-angiogenetic protein for the production of a medicament for inducing or inhibiting angiogenesis.

The medicament may be adapted to various forms of administration and may comprise further additives and/or excipients, such as, for example, carriers, stabilisers, preservatives and/or colourings. Examples of forms of administration are transplants, implants, infusion solutions, ointments, drops and/or tablets, preference being given to implants, transplants and infusion solutions.

The invention furthermore includes a cell or cells, especially fibroblasts, that has or have been genetically modified in such a manner that they express at least one angiogenetic or anti-angiogenetic protein. Those cells are suitable for the production of transplants and implants for insertion into the human or animal body for the purpose of inducing or inhibiting angiogenesis. Using a transplant or implant, greater success with the treatment, in the form of increased or decreased angiogenesis, is to be observed in comparison with conventional forms of administration of angiogenetic or anti-angiogenetic proteins.

The present invention further includes a method of treating a patient in whom angiogenesis is to be induced or inhibited, the method comprising the step of: (a) administering a therapeutically effective amount of the agent for inducing or inhibiting angiogenesis that comprises isogenic or autologous body cells that express at least one angiogenetic or anti-angiogenetic protein.

The therapeutically effective amount of the agent may be administered repeatedly, for example over several treatment cycles, with intervals between administrations.

The agent according to the invention is suitable, for example, in diabetics for the treatment of poorly healing ulcers or for the treatment of patients with peripheral arterial occlusion disease (PAOD) by increasing the formation of new blood vessels. It is also possible to use the agent in acute cases of inadequate perfusion (e.g. in the case of cardiac infarct). For the treatment of tumours, including especially those caused by a transformation of blood vessel forming cells, the agent according to the invention may be used anti-angiogenetically. Furthermore, the anti-angiogenetic agent may be used wherever local anti-angiogenetic treatment of tumours appears possible and appropriate (e.g. malignomas that have not yet metastasised).

The invention is described in more detail below with reference to practical embodiments and working examples and without limitation of the wording of the claims.

In a series of experimental studies, the inventor has discovered that after, for example, retroviral or adenoviral gene transfer of a nucleic acid coding for an angiogenetic or anti-angiogenetic protein into isogenic fibroblasts of rats (GMFB), the fibroblasts exhibit stable integration of, for example, the human gene PDGF-A in vitro. It was thereby possible to obtain in vitro up to a 560 times greater concentration of, for example, PDGF-AA compared with genetically non-modified fibroblasts (NMFB).

In addition, it was possible to show for the first time in vivo that GMFB as well as NMFB remain detectably vital after transplantation in a rat epigastric island flap model. The PDGF-AA produced by GMFB then resulted, under ischaemic conditions in the described model, in angiogenesis being manifested within 7 days of transplantation and thus in a significantly higher survival rate of ischaemically vulnerable flap tissue.

In a further experiment, it was possible to show that the described angiogenetic effects of PDGF-AA are ischaemia-dependent and cannot be produced under normal conditions in ischaemically non-vulnerable tissue. Although further studies, in which genetic cell manipulation was dispensed with and, instead, single bolus administration of, for example, VEGF165 and the selective VEGF165-antagonist sFLT-1 D1-D6 was used, generated angiogenetic effects, those effects fell far short of the results for PDGF-M effects caused by retrovirally modified fibroblasts. At the same time, it was possible to show that, clinically, the antagonist sFLT-1 D1-D6 produced in the laboratory of the inventor has an inhibitory effect on VEGF165.

The results found so far show that, in ischaemic conditions, functional angiogenesis brought about by temporary gene expression is most appropriate in vivo, in which case induction of VEGF165 and combined with sFLT-1 D1-D6 as selective antagonist for negative control is to take place first. Purely temporary gene expression in vivo may be achieved, for example, by selecting suitable inducible or repressible promoters in the expression construct. The advantage of such promoters resides in their ability to be regulated, so that the time at which the angiogenetic or anti-angiogenetic protein is expressed can be individually controlled from the outside by appropriate administration of substances that induce or repress the promoter.

Materials and Methods

Cell Cultures and Their Genetic Modification

Fibroblasts: fibroblast cultures obtained from autologous inbred rat strains (female Lewis-CRL rats), weight 200-215 g; Charles River Laboratories).
Virus-producing cell line: amphotrophic psi-CRIP packaging cell line descended from murine NIH-3T3 fibroblasts expressing the retroviral gene products gag, pol and env (R. Mulligan, Whitehead Institute of Biomedical Research, Cambridge, Mass. and W. Lindenmaier, Gesell-schaft für biotechnologische Forschung/Braunschweig). Transduction of the psi-CRIP pack-aging cell line with MFG plasmid DNA, cloning thereof and screening of the cell line producing the highest virion titres (J. R. Morgan, Shriners Burns Research Laboratories, Cambridge, Mass. and W. Lindenmaier, Gesellschaft für biotechnologische Forschung/Braunschweig)
Fibroblast cell culture medium: Dulbecco's modified Eagle's medium (DMEM, high-percentage glucose, L-gluamine, sodium pyruvate 100 mg/l from Gibco BRUUSA, FBS (foetal bovine serum) 10% (HtClone/USA), penicillin-streptomycin 100 IU/ml-100 μl/ml (Boehringer), BCS (bovine calf serurm) 10% (HtClone/USA)
PBS (phosphate-buffered saline solution) consisting of 138 mM NaCl, 2.7 M KCl, 8.1 mM Na₂HPO₄, 1.5 mM KH₂PO₄, sterilised through a 0.45 μm filter
EDTA—solution (named “Versene”) (=(ethylenedinitriol)-tetraacetic acid disodium salt, Boehringer): 5 mM dissolved in PBS and sterilised through a 0.45 μm filter
Trypsin solution (Trypsin 1-300, ICN Biochemicals), consisting of 0.1% D-dextrose (w/v) and trypsin 0.1% (w/v) in PBS at a pH of 7.5
Storage bottle for trypsin (25 ml, Wheaton Scientific)
Polybrene (Sigma/USA)
DMSO (dimethyl sulphoxide; Sigma-Aldrich; Irvine/England)
   Experimental Animals and Surgical Procedure
400 autologous Lewis rats (inbred strains; female) from Charles River Laboratories (Pittsfield N.H./USA). All animal experiments are carried out strictly in accordance with the relevant protocols of the Lübeck University Hospital.
Silastic sheet (0.0127 mm diameter; PharmElast, SF Medical Hudson Mass./USA)
Neck collars for rats as a protection against autocannibalism (Kent Scientific/Litchfield, Conn./USA)
Surgical instruments including microsurgery instruments
Ethyl ether (Fisher Scientific,. Fair Lawn, N.J./USA)
Ketamine (Ketanest 100 mg/ml; Fort Dodge Laboratories, Iowa/USA)
Xylazine (Rampun 20 mg/ml; Bayer Corporation, Kansas/USA).
Betadine
   Immunoassays and ELISA
ELISA (Enzyme Linked Immunosorbent Assay) (R&D Systems, Minneapolis/USA)
ELISA for VEGF-165, sFLT-1, PDGF-B and bFGF from the laboratory of Dr. Weich/GBF Braunschweig
   Histology Staining and Immunohistochemistry
Haematoxylin-eosin
Anti-human von Willebrand factor, IgG fraction (Sigma, St. Louis, Mo./USA)
FITC conjugate, anti-IgG (Sigma, St. Louis, Mo./USA)
   VEGF165-mRNA Analysis

The methods for VEGF-mRNA quantification are known per se (competitive RT-PCR, Northern blot).

Fibroblast Production

Autologous rat fibroblasts are cultured in 5 animals of the above-mentioned rat strain as subsequent carrier cells for the expression of the desired gene. For this, the animals are anaesthetised by intraperitoneal injection, for example of a combination of 0.05 mg/g ketamine (Ketanest 100 mg/ml; Fort Dodge Laboratories, Iowa/USA) and 0.0013 mg/g xylazine (Rampun 20 mg/ml; Bayer Corporation, Kansas/USA). The spontaneously breathing animals are shaved from the xiphoid to the inguinal region and placed on an operating table.

Body temperature is measured during each experiment by means of a digital rectal thermometer and is kept constant at 36-37° C. by means of a heating mat. After sterile washing-down of the operating area, an incision is made with a No. 10 scalpel from the xiphoid along the linea alba to the caudal, cutting through only the skin and the subcutaneous fatty tissue and leaving the fascia of the rectus musculature, in order subsequently to create an approximately 4×5 cm wound pocket bilaterally while carefully conserving the epigastric cutaneous vessels.

A Silastic membrane of equal size (Silastic is merely an example of a suitable material that after implantation brings about the formation of isogenic fibroblasts and otherwise has no further side-effects) (PharmElast; SF Medical) is introduced into that pocket and fixed in position by subcutaneous corner sutures (6-0 Ethilon). That membrane has a thickness of, for example, 0.0127 mm, is especially soft, extremely flexible and is supplied in sterile packaging. Before implantation of the sheet, it is washed under sterile conditions. After fixing of the sheet in situ, the wound is closed by intracutaneous 6-0 Ethilon suturing with countersinking of the corner knots.

The animals are kept under daily observation for 7 days, with free access to water and food, so that any problems in wound healing can be detected at an early stage. After that period, the animals are anaesthetised as described above and the Silastic membrane, which, as a foreign body, should have stimulated local fibroblast production, is removed together with the scar tissue which has formed around it. The animals are euthanised by means of an intra-peritoneal overdose of the mentioned anaesthesia mixture.

Fibroblast Separation and Culture

The surgically recovered fibroblast conglomerate is immediately stored in DMEM at 4° C. and subjected to as rapid as possible processing. The material is detached from the Silastic membrane under sterile conditions. All work on the fibroblast culture is carried out in a gene technology safety level 2 laboratory with air extraction at each sterile work station.

The material is then subjected to copious washing in a total of 10 plastics vessels each containing 10 ml of PBS (phosphate-buffered saline 0.9%). Each vessel can be sterile-sealed so that the fibroblast conglomerate can be vigorously shaken in the PBS for 10×60 seconds. After being washed twice, the tissue is taken out once more in order to remove the last remnants of connective tissue and pieces of Silastic, which have become demarcated, while taking sterile precautions.

The material is then placed in a sterile 25 ml monovette and enzyme-treated with 5 ml of trypsin and 5 ml of EDTA for 5 minutes to detach the fibroblasts from the collagen network. The cells are filtered through sterile gauze and then washed in DMEM with 20% FBS to neutralise the trypsin activity. The suspension so obtained is centrifuged at 800 rev/min for 5 minutes. The fibroblasts are deposited at the bottom and are mixed up with 10 ml of DMEM.

20 μl of the suspension are counted in a cell counter (haemocytometer) and the supernatant is subsequently removed by pipetting. The cells are then cultured in incubation chambers having a base surface area of 75 cm², with a seeding of 5×10⁴ cells/cm². Culturing is carried out in a medium composed of DMEM to which 100 μg/ml streptomycin, 100 IU/ml penicillin, 3 μl/ml amphotericin, 5% FBS and 10 μg/ml ascorbic acid have been added, which is added to the nutrient medium daily. The nutrient medium itself is changed every 3 days since the fibroblasts divide on average once every 16-18 hours and have a corresponding energy metabolism.

When the fibroblasts almost attain confluence, cell separation is carried out again. The cells obtained in that manner may subsequently be re-sown for further culturing of new cells or alternatively may be preserved and frozen. This is done by cell separation and subsequent suspension in DMEM with 10% FBS and appropriate addition of antibiotics. When CRIP cells are preserved, however, they are suspended with BCS medium. As a cryoprotection agent, the substance DMSO is admixed with the medium in a mixing ratio of 1:10. Each ml of medium should then contain between 1×10⁶ cells. Containers are then filled with 1-2 ml of medium each and frozen for 24 hours at −20° C. The following day, the containers are then deep-frozen at −80° C. in order to remain in turn, 24 hours later, in liquid nitrogen at −196° C. This staged freezing process avoids the formation of ice crystals in the suspension and consequent cell damage.

Production of Recombinant Retroviruses and Adenoviruses and Gene Transfer Into the Fibroblast Cultures

All of the gene technology work is carried out in accordance with the corresponding gene technology laboratory protocols in the safety level 1 and 2 areas provided for the purpose and is approved by the appropriate authority for gene technology safety.

The first step in the gene transfer consists of producing a recombinant virus that codes for the gene to be transferred. In these experiments, a cDNA that codes for the protein of interest is amplified by PCR (polymerase chain reaction). The corresponding primers produce, for example, a BspH1 locus at the translation start codon and, for example, a BamH1 locus at the translation stop codon. The PCR product can then be isolated by cleaving the gene product at the mentioned sites and inserting the gene obtained into the Nco1/BamH1 loci of a viral vector called MFG. Successful transfer is then verified by DNA sequencing.

That vector (MFG-plasmid DNA) originates, for example, from the murine Maloney leukaemia virus and does not itself contain any viral genes other than those necessary for transcription, packaging, reverse transcription, integration and expression of the viral vector with the modifying gene present therein. In order to be able to produce virions that are capable of genetically anchoring that vector in target cells, the vector has to be integrated into a special packaging cell line which originates, for example, from murine 3T3 fibroblasts. Transduction of the vector into the corresponding packaging cell line is facilitated by the addition of calcium phosphate since, by that means, the cell membranes of the packaging cell line temporarily become porous and provide the viral vector with easier access to the cell. This packaging cell line (Psi-CRI) was specifically produced to supply the retroviral proteins pol, env and gag which in turn are able to make virions that code for and transfer the vector with the modifying gene present therein. The packaging cell line itself is not able to make any viruses that correspond to “wild type” replicants and that are therefore virulent. Instead, it transcribes the DNA of the recombinant viral vector into RNA which is then integrated into the RNA of the virion. The Psi-CRP packaging cell line then secretes the virion, that is to say, the recombinant virus together with modifying gene, into the cell medium. Transfection of the target cells is effectively achieved at a quantity of 1.0-10.0 mill. virions/ml of medium. For that reason, every transfected cell line is examined for the highest titre production in order for it then to be selected for the gene transfer. For the adenoviral gene transfer, a padcos46 RESeGFP (39155 bp) is used as the cosmid vector fragment and is equipped with the corresponding desired gene sequence. The vector may, of course, be constructed in such a way that gene expression occurs only upon simultaneous administration of a further substance. By that means, time-controlled expression is possible. Suitable vectors having so-called “on/off” gene functions are known in the prior art.

For the transduction, the fibroblasts obtained are sown in incubation chambers of 75 cm² base surface area and with a medium composed of DMEM to which 100 μg/ml streptomycin, 100 IU/ml penicillin, 3 μl/ml amphotericin and 5% FBS have been added. Sowing of the fibroblasts is done at a low density (5×10⁵ cells) in order to obtain as great as possible effectiveness of the transduction.

On the following day, when all of the fibroblasts have made contact with the base surface of the incubation chamber and are slowly spreading, a medium change is carried out with medium from the Psi-CRIP cell line. In that medium there are 1.0-10.0 mill. virions/ml of medium which have been freshly pipetted from the medium of the packaging cell line (Psi-CRIP). The medium is for that purpose first filtered through a pore filter having a pore diameter of 0.45 μm to free it from cell debris and possible contaminants. The substance Polybrene is then admixed with the medium, in a concentration of 8 μg/ml.

Polybrene, like protamine and DEAE-dextran, is a cationic polymer, consisting of 1,5-dimethyl-1,5-diazadecamethylene polymethobromide, and produces its transfection-assisting effect by being adsorbed onto the virus particles and similarly also onto the surface of the target cell so as thereby to weaken the electrostatic repulsion forces of those two negatively charged materials. To obtain sufficiently large quantities of virus particles, the cells of the Psi-CRIP cell line must already be confluent and a medium change has to be carried out on the day before the transduction in order to ensure as great as possible a number of active viruses/ml of medium. At 37° C. in the incubator, the viruses have an average half-life of 6-8 hours. By lowering the incubator temperature to 32° C., the life of the viruses can be prolonged by up to ten times and hence the effectiveness of the transduction can be considerably increased.

When the recombinant viruses come together with target cells in the medium, the virion is bound to the cell surface of the fibroblasts by special receptors and releases the packaged RNA genome into the interior of the cell. This is reverse-transcribed and the resulting DNA enters the cell nucleus where it is stably integrated into the genome of the target cell. That integrated copy of the recombinant viral vector with the modifying gene is passed on to the daughter cells like any other autosomal gene. In addition, stable, regular expression of the gene takes place, with the result that the target cells then secrete large quantities of the desired protein.

When adenoviral vectors are used, an only temporary gene expression is to be expected, which will be eliminated from the cell genome again after a number of cell generations. The medium should be left together with the target cells for 24 hours so that the transduction process is completed in all the cells.

The virus-containing medium may also be preserved in order to be used for transduction of fibroblasts at a later date. For that purpose, the medium is pipetted off and shock-frozen on dry ice until it assumes a yellowish coloration. The medium is then stored at −80° C. A loss of 30-50% of the viruses as a result of that procedure must be expected, however.

The growth of the genetically modified fibroblasts compared with untreated fibroblasts is studied by sowing batches of 5×10⁵ cells each on 60 mm diameter petri dishes and counting the cells at 12 hour intervals for a total of 4 days following the above-mentioned procedure. From this, it is possible to draw conclusions on the biological activity of the secreted protein, since, with the exception of sFLT1 D1-D6, the substances used also have an autocrine mitogenic effect, that is to say, the fibroblasts which themselves secrete the protein are stimulated to cell division. The number of fibroblasts is then measured under a fluorescence microscope by means of a counting chamber and the vitality of the cells is determined by Evans blue and compared with the controls which consist of untreated fibroblasts.

In addition, protein expression is determined in vitro by means of ELISA techniques. Significantly greater protein production by the genetically modified fibroblasts is expected in that case.

After being quantified, the cell populations prepared in that manner are transported in medium in order to carry out the operation which then follows.

Operation

2 subgroups (I and II) each comprising 200 animals are formed. Each subgroup is in turn divided into 5 sub-subgroups (I.I-I.V, II.I-II.V) each comprising 40 animals. Since 4 different factors are to be tested (VEGF 165, VEGF 165+sFLT-1, PDFG-B and bFGF), each sub-subgroup needs to be split in turn into 4 subgroups each comprising 10 animals. The body weight of the animals is regularly determined during the days of the experiment by means of digital scales.

In group I, one week before the actual operation each flap is marked on the ether-anaesthetised animal in an area of 7×7 cm and treatment of the flap is already carried out at that time.

Group I.I receives in the marked flap region a subcutaneous injection of 10 mill. genetically modified fibroblasts (GMFB) in 2 ml of DMEM with 10% FBS. The injection itself is carried out using a sterile 2 ml syringe having a 0.4 mm diameter steel cannula into the Panniculus carnosus between the outer fascial sheet of the stomach wall and subcutis. Group I.II receives in the marked flap region a subcutaneous injection of 10×10⁶ non-modified fibroblasts (NMFB) suspended in 2 ml of DMEM with 10% FBS. Group I.III receives a subcutaneous injection of 2 ml of DMEM with 10% FBS without the addition of cells. Group I.IV receives a subcutaneous injection of 2 ml of NaCl 0.9% without the addition of cells. Exactly one week later, surgical elevation of the flaps prepared in that manner is carried out.

In group II, flap treatment is carried out on the day the flap is elevated. Group I.V and group II.V are treated in the same way as groups I.I and II.I and are used for long-term experiments. Those groups of animals are sacrificed only after an observation period of 6 months (5 animals in each case) and 12 months (5 animals in each case).

The surgical procedure is identical in all subgroups. The animals are anaesthetised by intraperitoneal injection of a combination of 0.05 mg/g of rat of ketamine (Ketanest 100 mg/ml; Fort Dodge Laboratories, Iowa/USA) and 0.0013 mg/g of rat of xylazine (Rampun 20 mg/ml; Bayer Corporation, Kansas/USA). The spontaneously breathing animals are shaved from the xiphoid to the inguinal region and placed on an operating table. Body temperature is measured during each experiment by means of a digital rectal thermometer and is kept constant at 36-37° C. by means of a heating mat.

In each animal, a standardised epigastric flap is elevated with the dimensions 7×7 cm. First, the base of the flap is nicked, the femoral vessels on both sides are selected and then the flap including the skin and subcutis at the two inferior epigastric vascular nerve bundles is completely elevated so that the perfusion of the flap continues to be ensured solely by those vascular pedicles. The superior epigastric vascular pedicles are severed following ligature by means of 6-0 Ethilon sutures. For each flap, the left-side vascular nerve bundle is also severed under 2 6-0 Ethilon ligatures so that the flap is then nourished merely via the right-side pedicle vessels.

Group II.I and group II.V receive in the marked flap area a subcutaneous injection of 10×10⁶ genetically modified fibroblasts (GMFB) in 2 ml of DMEM with 10% FBS. The injection itself is made in the same way as already described for group I.I. Group II.II receives in the marked flap area a subcutaneous injection of 10×10⁶ non-modified fibroblasts (NMFB) suspended in 2 ml of DMEM with 10% FBS. Group II.III receives a subcutaneous injection of 2 ml of DMEM with 10% FBS without the addition of cells. Group II.IV receives a subcutaneous injection of 2 ml of NaCl 0.9% without the addition of cells. Each flap is then sewn back into its wound bed. For that purpose, first 4 corner sutures and 2 sutures in the median line are made with a 6-0 Ethilon suture and then the entire flap is sewn in intracutaneously by a running 6-0 Ethilon suture with countersinking of the knots.

Post-operatively, all the animals are given a neck collar (Kent Scientific) to protect them from autocannibalism. Exactly one week after that surgery, the animals in groups I.I-I.IV and II.I-II.IV are operated on for a last time. The flaps are recorded on a plastics sheet, divided planimetrically into mm², according to their proportion of vital and necrotic flap tissue for subsequent computer-controlled image analysis. After subsequently elevating the flaps once more, the flap specimens are finally removed in their entirety together with the underlying muscle layer for further histological, immunohistochemical and mRNA-analytical investigation. Finally, the test animals are sacrificed by an intraperitoneal overdose of ketamine.

6 months and 12 months after cell transplantation, histological and immunohistochemical evaluation of the long-term results is made in the animals of groups I.V and II.V in order to be able to study in more detail the permanent sequelae of genetic manipulation in the tissue.

All of the operations are performed in gene technology safety level 1 areas provided for the purpose by LUH.

Histology, Immunohistochemistry and Tissue Extraction

Histological processing of the specimens is carried out after fixing in formaldehyde and staining in haematoxylin/eosin. The specimens are embedded beforehand in paraffin, cut into sections of 5 μm thickness using a microtome blade, fixed in specimen plates and subjected to appropriate staining. Staining of the specimens is carried out separately after haematoxylin/eosin as the primary stain and the counterstain in immunohistochemistry.

10 further animals from groups I and II whose flap tissue had been treated with GMFB are sacrificed after 6 and 12 months, respectively, and the angiogenetically modified tissue still remaining is subjected to histological examination.

Immunohistochemically, staining by means of immunoperoxidase for factor VIII (von Wille-brand factor) with rabbit antiserum for staining endothelial cells and chloroacetate esterase staining for visualising polymorphonuclear cells are carried out. In addition, staining is carried out to detect the secreted protein in the flap tissue.

The freshly removed tissue is examined for its ability to produce produced protein by means of mRNA analysis by quantitative PCR analysis in order to obtain an indication as to the amount and duration of protein production by the GMFB.

The production of autologous cells for re-transplantation in the same donor organism is carried out according to exactly the same methodological specifications as those described previously.

### LITERATURE REFERENCES

1. Genetically modified fibroblasts induce angiogenesis in the rat epigastric island flap Machens H G, Morgan J R, Berthiaume F, Stefanovich P and Berger A in: Biological matrices and tissue reconstruction (ed.: Stark, Horch, Tanczos) pp. 53-59 Springer Verlag/Berlin Heidelberg New York (1997) ISDN 3-54063863-6
2. Genetically modified fibroblasts induce angiogenesis in the rat epigastric island flap Machens H G, Morgan J R, Berthiaume F, Stefanovich P, Reimer R and Berger A Langenbeck's Arch Surg 383: 345-350 (1998)
3. Genetically modified fibroblasts induce angiogenesis in the 3×6 cm rat epigastric island flap Machens H G, Morgan J, Weich H E, Berthiaume F, Stefanovich P, Berger A Eur J Plast Surg 22: 203-209 (1999)
4. Genetically modified fibroblasts induce angiogenesis in the 3×6 cm rat epigastric island flap—authors' reply. Machens H G, Morgan J, Weich H E, Berthiaume F, Stefanovich P, Berger A Eur J Plast Surg 22: 211-212 (1999)
5. Gentherapeutische Techniken und Anwendungsmöglichkeiten in der Plastischen Chirurgie Machens H G, Morgan J R, Mailänder P Focus MUL 17: 3-10 (2000)
6. Gentherapeutische Möglichkeiten in der Plastischen Chirurgie Machens H G, Morgan J R, Sachse C, Berger A, Mailänder P Chirurg 70: 176-181 (2000)

1. An agent for inducing or inhibiting angiogenesis, which comprises isogenic or autologous body cells that express at least one angiogenetic or anti-angiogenetic protein.

2. An agent according to claim 1, wherein the at least one angiogenetic protein is selected from PDGF-A (platelet derived growth factor A), PDGF-B (platelet derived growth factor B), VEGF (vascular endothelial growth factor), bFGF (basic fibroblast growth factor), TGFbeta (tumour growth factor beta), angiopoetin I and angiopoetin.

3. An agent according to claim 1, wherein the at least one anti-angiogenetic protein is selected from VEGFR-1 (vascular endothelial growth factor receptor 1), angiostatin, endostatin and receptor blockers for VEGFR1 and VEGFR2.

4. A method for the production of an agent according to claim 1, in which
a) the formation of isogenic or autologous cells is induced in a body by implantation of biologically inert material,
b) the cells formed in step a) are extracted from the body,
c) the cells obtained in step b) are genetically modified in such a manner that in the case of a desired induction of angiogenesis they express at least one angiogenetic protein or in the case of a desired inhibition of angiogenesis they express at least one anti-angiogenetic protein.

5. A method according to claim 4, wherein the cells obtained in step c) express an angiogenetic protein selected from PDGF-A (platelet derived growth factor A), PDGF-B (platelet derived growth factor B), VEGF (vascular endothelial growth factor), bFGF (basic fibroblast growth factor), TGFbeta (tumour growth factor beta), angiopoetin 1 and angiopoetin.

6. A method according to claim 4, wherein the cells obtained in step c) express an anti-angiogenetic protein selected from VEGFR-1 (vascular endothelial growth factor receptor 1), angiostatin, endostatin and receptor blockers for VEGFR1 and VEGFR2.

7. The use of an agent according to claim 1 for inducing or inhibiting angiogenesis.

8. The use of an agent according to claim 1 for inducing or inhibiting angiogenesis in which the agent is introduced into isogenic or autologous tissue of the body in which angiogenesis is to be induced or inhibited.

9. The use of an agent produced by a method according to claim 4 for inducing or inhibiting angiogenesis.

10. The use of an agent produced by a method according to claim 4 for inducing or inhibiting angiogenesis in which the agent is introduced into isogenic of autologous tissue of the body in which angiogenesis is to be induced or inhibited.
